(19) United States
(12) Patent Application Publication
(10) Pub. No.: US 2013/0131033 A1
(43) Pub. Date: May 23, 2013

(54) PHARMACEUTICAL COMPOSITION FOR TREATMENT OF RESPIRATORY TRACT INFECTIONS

(71) Applicant: Mayank Jasvantbhai Shah, Mumbai (IN)

(72) Inventor: Mayank Jasvantbhai Shah, Mumbai (IN)

(73) Assignee: TOYOCHEM LABORATORIES, Mumbai (IN)

(21) Appl. No.: 13/809,922

(22) PCT Filed: Jan. 6, 2011
(86) PCT No.: PCT/IN2011/000008

§ 371 Date: Jan. 14, 2013

(30) Foreign Application Priority Data
Jan. 13, 2010 (IN) 108/MUM/2010

(51) Int. Cl. A61K 31/496; A61K 31/43

(52) U.S. Cl. CPC A61K 31/496; A61K 31/43
USPC 514/197; 264/109

(57) ABSTRACT

The present invention relates to a pharmaceutical composition comprising ciprofloxacin or salts of thereof and amoxycillin or salts of thereof and other pharmaceutical excipients. The present invention provides more reliable effect against all bacteria, including the most commonly causative streptococci, responsible for especially respiratory tract infections. The invention makes up for the weakness of ciprofloxacin against streptococci.

The present invention relates to a pharmaceutical composition for the treatment of respiratory tract infections. This composition comprises ciprofloxacin or salts of thereof and amoxycillin or salts of thereof.

Ciprofloxacin has reliable effectiveness against all bacteria responsible especially for upper respiratory tract infections (URTI) and lower respiratory tract infections (LRTI)—except against streptococci species, which is one of the commonest pathogen for respiratory tract infections (RTIs). In view of amoxycillin's continual reliability for streptococci, the combination of these two antimicrobials offers an effective option as preferred first line for empiric therapy of RTIs.

### BACKGROUND OF THE INVENTION

Ciprofloxacin is a synthetic antibiotic, belonging to a group called fluoroquinolones. As far as streptococci are concerned, in recent years, these are inherently less susceptible to especially older fluoroquinolones. {Kaneko A et al. J Antimicrob Chemother 2000; 45: 771-775; Kaneko A et al. Chemotherapy (Tokyo) 1994; 42: 94-100; Coyle EA et al. Antimicrob Ag Chemother 2001; 45: 6: 1654-1659} A major mechanism of resistance to fluoroquinolones in Gram-positive bacteria such as Streptococcus pneumoniae is target modification; mutations in the quinolone resistance determining region (QRDR) of the gyrA, parC and parE genes, resulting in decreased affinity for the quinolones. {Kaneko A et al. J Antimicrob Chemother 2000; 45: 771-775; Margerrison E E et al. J of Bacteriology 1992; 174: 1596-1603; Ferrero L et al. Antimicrob Ag Chemother 1995; 39: 1554-1558; Jorgensen J H et al. Antimicrob Ag Chemother 1999; 43: 329-334}

Besides being less susceptible, resistance of Streptococcus pneumoniae to ciprofloxacin is the most amongst quinolones, {Alou L et al. Antimicrob Ag Chemother 2001; 45: 10: 2955-2957} and as many as 15% of these bacteria are possibly resistant to it. {Deshpande L M et al. Diagn Microbiol Infect Dis 2006; 54: 3: 157-164}). In India, the resistance of streptococci to fluoroquinolones is more likely to be prevalent since ciprofloxacin and other similar agents are more used for children. This is because the reservoir for streptococci is children. (http://www.annals.org/cgi/content/full/129/11_Part_{—}1/908. As read on 15Nov. 2008)

Amoxycillin is a moderate-spectrum, bactericidal, β-lactam antibiotic used to treat bacterial infections caused by susceptible microorganisms. Amoxycillin is advocated as preferred agent for URTI. {Korner J R et al. BMJ 1994; 308: 191-192} With the ever-changing pattern of bacterial resistance, use of conventional doses amoxycillin in LRTI to eradicate H influenzae is disastrous. {Spiteri M. BMJ 1994; 308: 657-658; Powell M et al. J Antimicrob Chemother 1991; 28: 249-259}) In fact, amoxycillin is even lesser effective than ampicillin against H influenzae. {The Use of Antibiotics. Kucers A et al (Eds) 5^{th} Ed 1997; Butterworth/Heinemann pp 134-144; Kosmidis J et al. Brit J Clin Pract 1972; 26: 341}

### Objects of the Present Invention

The main object of the present invention is to provide a pharmaceutical composition of ciprofloxacin and amoxycillin or salts of thereof that provides a more reliable effect against all bacteria responsible for especially RTIs.

Further object of the invention is to give tolerated dosage of these two antibiotics.

Another object of the invention is to make up for the weakness of ciprofloxacin against streptococci.

Yet an object of the invention is to provide a composition that shows synergistic effect with no evidence of antagonism.

It is yet another object of the present invention to provide a simple process for preparing oral solid dosage forms comprising pharmaceutically effective amounts of ciprofloxacin and amoxycillin or salts of thereof.

### SUMMARY OF THE INVENTION

The present invention relates to a pharmaceutical composition comprising ciprofloxacin and amoxycillin. In the invention ciprofloxacin or salts of thereof and amoxycillin or salts of thereof are used. Ciprofloxacin or salts of thereof is used in 100 to 750 mg/tablet and amoxycillin or salts of thereof are used in 200 to 1000 mg/tablet.

### DETAIL DESCRIPTION

Ciprofloxacin is indicated for treatment of acute uncomplicated cystitis in females, urinary tract infections (UTI), chronic bacterial prostatitis, LRTI, acute sinusitis, skin and skin structure infections (SSTI), bone and joint infections, complicated intra-abdominal infections, infectious diarrhea, enteric fever, uncomplicated cervical and urethral gonorrhea due to susceptible pathogens. Ciprofloxacin is bactericidal. Its mode of action depends upon blocking bacterial DNA replication by binding itself to an enzyme called DNA gyrase, thereby inhibiting the unwinding of bacterial chromosomal DNA during and after the replication. Ciprofloxacin is a broad spectrum antibiotic that is active against both Gram positive and Gram negative bacteria.

Amoxycillin, a penicillinase-susceptible semi-synthetic penicillin, is a close chemical and pharmacological relative of ampicillin. The drug is stable in acid and is designed for oral use. It is more rapidly and completely absorbed from the gastrointestinal tract than is ampicillin, which is the major difference between the two. The antimicrobial spectrum of amoxycillin is essentially identical to that of ampicillin, with the important exception that of ampicillin that amoxycillin appears to be less effective than ampicillin for shigellosis. (P. A. Willium, Chapter 45 Antimicrobial Agents; Goodman & Gilman's The Pharmacological Basis of Therapeutics; 10^{th} Edition; McGraw-Hill Medical Publishing Division; Page: 1202) Amoxycillin is usually the drug of choice within the class because it is better absorbed, following oral administration, than other beta-lactam antibiotics. It is susceptible to degradation by β-lactamases-producing bacteria, and so may be given with clavulanic acid to decrease its susceptibility. Amoxycillin acts by inhibiting the synthesis of bacterial cell wall. It inhibits cross-linkage between the linear peptidoglycan polymer chains that make up a major component of the cell wall of Gram-positive bacteria.

In the invention ciprofloxacin or salts of thereof and amoxycillin or salts of thereof are used. Ciprofloxacin or salts of thereof is used in 100 to 750 mg/tablet and amoxycillin or salts of thereof is used in 200 mg to 1000 mg/tablet.

Further in present invention other pharmaceutical excipients are used. These excipients include binders, glidants, diluents, lubricants, disintegration agents and solvent.

Embodiment of the invention relates to the process for preparation of pharmaceutical formulation which involves the following steps:

(a) All the ingredients are weighed as per the required quantity.

(b) Ciprofloxacin HCl, starch I.P. (⅔^{rd} quantity) and diluents are passed through 40# sieve and mixed for 15 minutes.

(c) Starch paste is prepared by adding required quantity of water (125 ml for 500 tablets) in the remaining amount of starch (⅓^{rd} Quantity).

(d) The mixture obtained in step (b) is granulated with the prepared starch paste.

(e) The granules as obtained in step (d) are semi dried in tray drier at 65° C.

(f) The semi-dried granules are removed and passed through 12# sieve

(g) Further, granules are dried in tray drier at 65° C. till the LOD (Loss on Drying) is less than 3%.

(h) The dried granules are passed through 16# sieve.

(i) The weighed quantity of amoxycillin trihydrate I.P. is passed through 60# sieve and mixed with granules obtained in step (h) for 15 minutes.

(j) Glidant, lubricant, disintegration agent, super disintegration agent and binder are passed through 60# sieve and mixed with the blend obtained in step (i).

(k) The lubricated granules are compressed in to tablet.

The invention is illustrated more in detail in the following examples. The examples describe and demonstrate embodiments within the scope of the present invention. These examples are given solely for the purpose of illustration and are not to be construed as limitations of the present invention, as many variations thereof are possible without departing from the spirit and scope.

### EXAMPLE 1

| Sr. No. | Ingredient | Amount/Tablet | |
|---|---|---|---|
| 1 | Ciprofloxacin hydrochloride IP | 148.10 | mg |
| | Equivalent to Ciprofloxacin IP | 125 | mg |
| 2 | Amoxycillin Trihydrate I.P. | 229.33 | |
| | Equivalent to Amoxycillin | 200 | mg |
| 3 | Starch I.P. | 20 | mg |
| 4 | Micro crystalline cellulose I.P. | 15 | mg |
| 5 | Talcum IP | 7.5 | mg |
| 6 | Magnesium stearate IP | 7.5 | mg |
| 7 | Aerosil 200 IP | 3 | mg |
| 8 | Sodium starch glycolate IP | 6 | mg |
| 9 | Polyvinyl pyrrolidone K-30 IP | 3.75 | mg |

| | | |
|---|---|---|
| 10 | Water | Q.S. |

### Process

(i) All the ingredients are weighed as per the required quantity.

(ii) Ciprofloxacin HCl, starch I.P. (⅔^{rd} quantity) and micro crystalline cellulose I.P. are passed through 40# sieve and mixed for 15 minutes.

(iii) Starch paste is prepared by adding required quantity of water (125 ml for 500 tablets) in the remaining amount of starch (⅓^{rd} Quantity).

(iv) The mixture obtained in step (ii) is granulated with the prepared starch paste.

(v) The granules as obtained in step (iv) are semi dried in tray drier at 65° C.

(vi) The semi-dried granules are removed and passed through 12# sieve

(vii) Further, granules are dried in tray drier at 65° C. till the LOD (Loss on Drying) is less than 3%.

(viii) The dried granules are passed through 16# sieve.

(ix) The weighed quantity of amoxycillin trihydrate I.P. is passed through 60# sieve and mixed with granules obtained in step (viii) for 15 minutes.

(x) Talcum, magnesium stearate, aerosil 200, sodium starch glycolate and PVP K-30 are passed through 60# sieve and mixed with the blend obtained in step (ix).

(xi) The lubricated granules are compressed in to tablet.

### EXAMPLE 2

| Sr. No. | Ingredient | Amount/Tablet | |
|---|---|---|---|
| 1 | Ciprofloxacin hydrochloride IP | 296.20 | mg |
| | Equivalent to Ciprofloxacin IP | 250 | mg |
| 2 | Amoxycillin Trihydrate I.P. | 458.66 | mg |
| | Equivalent to Amoxycillin | 400 | mg |
| 3 | Starch I.P. | 40 | mg |
| 4 | Micro crystalline cellulose I.P. | 30 | mg |
| 5 | Talcum IP | 15 | mg |
| 6 | Magnesium stearate IP | 15 | mg |
| 7 | Aerosil 200 IP | 6 | mg |
| 8 | Sodium starch glycolate IP | 12 | mg |
| 9 | Polyvinyl pyrrolidone K-30 IP | 7.5 | mg |

| | | |
|---|---|---|
| 10 | Water | Q.S. |

| | | |
|---|---|---|
| Total weight per tablet | 880.36 | mg |

### Process

The process is same as mentioned in Example 1.

### EXAMPLE 3

| Sr. No. | Ingredient | Amount/Tablet | |
|---|---|---|---|
| 1 | Ciprofloxacin hydrochloride IP | 592.41 | mg |
| | Equivalent to Ciprofloxacin IP | 500 | mg |
| 2 | Amoxycillin trihydrate I.P. | 917.33 | mg |
| | Equivalent to Amoxycillin | 800 | mg |
| 3 | Starch I.P. | 30 | mg |
| 4 | Talcum IP | 30 | mg |
| 5 | Magnesium stearate IP | 25 | mg |
| 6 | Aerosil 200 IP | 8 | mg |
| 7 | Sodium starch glycolate IP | 15 | mg |
| 8 | Polyvinyl pyrrolidone K-30 IP | 20 | mg |

| | | |
|---|---|---|
| 9 | Water | Q.S. |

| | | |
|---|---|---|
| Total weight per tablet | 1637.74 | mg |

### Process

(i) All the ingredients are weighed as per the required quantity.

(ii) Ciprofloxacin HCL and starch I.P. (⅔^{rd} quantity) are passed through 40# sieve and mixed for 15 minutes.

Other steps are same as mentioned in Example 1.

### In Vitro Antibacterial Sensitivity Study

An in vitro antibacterial sensitivity study for a combination of amoxycillin and ciprofloxacin against Streptococcus pyogenes (MTCC 442) and Streptococcus pneumoniae (MTCC 2672) was conducted at MICRO THERAPEUTIC RESEARCH LABS PVT. LTD. at its pre-clinical facility based at Chennai by Study No.: AMOX-538-09. The study report was generated on 1Mar. 2010. The study was conducted as per the approved study protocol.

The study was conducted to determine the antibacterial sensitivity of ciprofloxacin, amoxycillin and their combination (1:1.6) against Streptococcus pyogenes (MTCC 442) & Streptococcus pneumoniae (MTCC 2672) by using two methods: (a) Broth Macrodilution Method (b) Disc Diffusion Method. The test substances used were three formulations, Formulation I, Formulation II and Formulation III prepared in accordance with Example 1, Example 2 and Example 3 respectively. Amoxycillin powder and Ciprofloxacin HCl powder used as reference standards were procured from HiMedia Laboratories. The preparations and the method of the study were as per the approved protocol.

### Results & Interpretation

### (a) Broth Macrodilution Method

The MIC of test substance formulation against Streptococcus pyogenes (MTCC 442) and Streptococcus pneumoniae (MTCC 2672) is as displayed in Table 1.

TABLE 1

| | MIC (μg/ml) against | MIC (μg/ml) against |
|---|---|---|
| Drug | Streptococcus pyogenes | Streptococcus pneumoniae |
| Amoxycillin | 0.031 | 0.063 |
| Ciprofloxacin | 0.313 | 0.625 |
| Formulation I | Amoxycillin-0.016 | Amoxycillin-0.031 |
| | Ciprofloxacin-0.010 | Ciprofloxacin-0.020 |
| Formulation II | Amoxycillin-0.016 | Amoxycillin-0.016 |
| | Ciprofloxacin-0.010 | Ciprofloxacin-0.010 |
| Formulation III | Amoxycillin-0.016 | Amoxycillin-0.016 |
| | Ciprofloxacin-0.010 | Ciprofloxacin-0.010 |

### (b) Disc Diffusion Method

The zone of inhibition for all test substance formulations and reference substance for Streptococcus pyogenes and Streptococcus pneumoniae have been tabulated in Table 2.

TABLE 2

| | | | Zone Diameter | Zone Diameter |
|---|---|---|---|---|
| Sr. | | | Range (mm) | Range (mm) |
| No | Sample | Dose μg/disc | S. pyogenes | S. pneumoniae |
| 1. | DMSO | 1% v/v | Nil | Nil |
| | (Negative | | | |
| | Control) | | | |
| 2. | Reference | 8 | 32-33 | 33-36 |
| | standard | 16 | 34-36 | 34-36 |
| | Amoxycillin | 32 | 37-39 | 37-38 |
| 3. | Reference | 5 | 20-22 | 18 |
| | Standard | 10 | 22-23 | 18-21 |
| | Ciprofloxacin | 20 | 23-26 | 20-23 |
| | HCl | | | |
| 4. | Formulation I | 8 μg Amoxycillin + | 36 | 38 |
| | | 5 μg Ciprofloxacin | | |
| | | 16 μg Amoxycillin + | 36 | 39 |
| | | 10 μg Ciprofloxacin | | |
| | | 32 μg Amoxycillin + | 40 | 39-40 |
| | | 20 μg Ciprofloxacin | | |
| 5. | Formulation II | 8 μg Amoxycillin + | 36 | 38 |
| | | 5 μg Ciprofloxacin | | |
| | | 16 μg Amoxycillin + | 36-37 | 38-39 |
| | | 10 μg Ciprofloxacin | | |
| | | 32 μg Amoxycillin + | 38-40 | 40 |
| | | 20 μg Ciprofloxacin | | |
| 6. | Formulation III | 8 μg Amoxycillin + | 36 | 37 |
| | | 5 μg Ciprofloxacin | | |
| | | 16 μg Amoxycillin + | 36-38 | 37-38 |
| | | 10 μg Ciprofloxacin | | |
| | | 32 μg Amoxycillin + | 39-40 | 39 |
| | | 20 μg Ciprofloxacin | | |

The sensitivity of the test substances and the reference standards were interpreted as per the CLSI zone diameter interpretive standards shown in Table 3.

TABLE 3
Zone Diameter Interpretive Standards for Streptococcus sp.

| | Zone Diameter, nearest whole mm | | |
|---|---|---|---|
| Antibiotic | Susceptible (S) | Intermediate (I) | Resistant (R) |
| Amoxycillin | ≧26 | 19-25 | ≦18 |
| Ciprofloxacin | ≧21 | 16-20 | ≦15 |

### CONCLUSION

It has been found that the test organisms Streptococcus pyogenes (MTCC 442) and Streptococcus pneumoniae (MTCC 2672) are susceptible to the combination formulation of amoxycillin and ciprofloxacin as well as to individual drugs. For both organisms, MIC of ciprofloxacin was higher than the MIC of amoxycillin. In contrast, the MIC of ciprofloxacin in combination formulations was lower when compared to the MIC of amoxycillin in combination formulations. Thus the formulation containing combination of amoxycillin and ciprofloxacin has lower MIC as compared to individual components.

Thus, it can be concluded that the combination formulation of amoxycillin and ciprofloxacin is more effective against Streptococcus pyogenes and Streptococcus pneumoniae than the individual components alone.

We claim:

1. A pharmaceutical composition for treatment of respiratory tract infection comprises ciprofloxacin or salts of thereof, amoxycillin or salts of thereof, starch and other pharmaceutical excipients.

2. The pharmaceutical composition as claimed in claim 1 wherein ratio of ciprofloxacin:amoxycillin is 1:1.6.

3. The pharmaceutical composition as claimed in claim 1 wherein 100 to 750 mg/tablet ciprofloxacin is used.

4. The pharmaceutical composition as claimed in claim 3 wherein more preferably 125 mg/tablet or 250 mg/tablet or 500 mg/tablet ciprofloxacin is used.

5. The pharmaceutical composition as claimed in claim 1 wherein 200 to 1000 mg/table amoxycillin is used.

6. The pharmaceutical composition as claimed in claim 5 wherein more preferably 200 mg/tablet or 400 mg/tablet or 800 mg/tablet amoxycillin is used.

7. The pharmaceutical composition as claimed in claim 1 wherein pharmaceutical excipients include binders, glidants, diluents, lubricants, disintegration agents and solvent.

8. The process of preparing a pharmaceutical composition for treatment of respiratory tract infection comprises the following steps:
(a) weighing all the ingredients;
(b) passing ciprofloxacin, starch I.P. (⅔^{rd} quantity) and diluents through 40# sieve and mixing for 15 minutes;
(c) preparing starch paste by adding water in the remaining amount of starch (⅓^{rd} Quantity);
(d) granulating the mixture obtained in step (b) with the prepared starch paste;
(e) semi-drying granules as obtained in step (d) in tray drier at 65° C.;
(f) removing semi-dried granules and passing through 12# sieve;
(g) further, drying granules in tray drier at 65° C. till the LOD (Loss on Drying) less than 3%;
(h) passing the dried granules through 16# sieve;
(i) passing weighed quantity of amoxycillin trihydrate I.P. through 60# sieve and mixing with granules obtained in step (h) for 15 minutes;
(j) passing glidant, lubricants, disintegration agent, super disintegration agent and binder through 60# sieve and mixing with the blend obtained in step (i);
(k) compressing lubricated granules in to tablet.

9. The pharmaceutical composition for treatment of respiratory tract infection herein described with reference to the foregoing description and examples.

10. The process for preparation of pharmaceutical composition for treatment of respiratory tract infection herein described with reference to the foregoing description and examples.
